(19) United States
(12) Patent Application Publication
(10) Pub. No.: US 2015/0351725 A1
(43) Pub. Date: Dec. 10, 2015

(54) ULTRASONIC DIAGNOSIS APPARATUS AND MEDICAL IMAGE PROCESSING APPARATUS

(71) Applicants: Kabushiki Kaisha Toshiba, Minato-ku (JP); Toshiba Medical Systems Corporation, Otawara-shi (JP)

(72) Inventors: Taku MURAMATSU, Nasushiobara (JP); Shouichi NAKAUCHI, Nasushiobara (JP); Katsuyuki TAKAMATSU, Yaita (JP); Nami FUJIMOTO, Nasushiobara (JP); Takashi MASUDA, Nasushiobara (JP)

(73) Assignees: Kabushiki Kaisha Toshiba, Minato-ku (JP); Toshiba Medical Systems Corporation, Otawara-shi (JP)

(21) Appl. No.: 14/830,394

(22) Filed: Aug. 19, 2015

Related U.S. Application Data
(63) Continuation of application No. PCT/JP2014/000828, filed on Feb. 18, 2014.

(30) Foreign Application Priority Data
Feb. 20, 2013 (JP) 2013-031197

(51) Int. Cl. A61B 8/08; A61B 8/00; A61B 8/14

(52) U.S. Cl. CPC A61B 8/5246; A61B 8/14; A61B 8/5207; A61B 8/466; A61B 8/4254; A61B 8/54

(57) ABSTRACT

An ultrasonic diagnosis apparatus according to an embodiment includes: an image data generation section generates two-dimensional ultrasonic images of a subject; an image display processing section processes the two-dimensional ultrasonic images to generate a three-dimensional image; a mark setting section sets a mark in a region of interest of the three-dimensional image; and a controller controls to perform predetermined processing uses an information of the mark, when the space region corresponding to the mark is scanned by an ultrasonic probe in rescanning operation for the subject.

### CROSS-REFERENCE TO RELATED APPLICATION

This application is a continuation-in-part of International Application No. PCT/JP2014/000828, filed on Feb. 18, 2014, which is based upon and claims the benefit of priority from the prior Japanese Patent Application No. 2013-31197, filed on Feb. 20, 2013, the entire contents of which are incorporated herein by reference.

### FIELD

Embodiments described below relate to an ultrasonic diagnosis apparatus and a medical image processing apparatus which are capable of displaying a three-dimensional image by performing rescanning for a region of interest.

### BACKGROUND

Conventionally, when using an ultrasonic probe with a position sensor to perform scanning by an ultrasonic diagnosis apparatus, an operator manually adjusts an angle and a direction of the probe while confirming an ultrasonic image being displayed in real time to thereby create and display three-dimensional image data of a target region.

However, when acquiring the three-dimensional image data for only the region of interest, the operator manually designates a scan start/end position for each scanning operation, thus exhibiting poor reproducibility of an image data collection start/end position. Further, when one region of interest is scanned from different directions, operation is performed based on only subjectivity of an operator, so that a similar image, if exists in the vicinity of the region of interest, may be mistaken for an image of the region of interest.

### BRIEF DESCRIPTION OF THE DRAWINGS

FIG. 1 is a block diagram illustrating a configuration of an ultrasonic diagnosis apparatus according to an embodiment;

FIGS. 2A to 2D are explanation views illustrating a basic operation of the ultrasonic diagnosis apparatus according to the embodiment;

FIG. 3 is a flowchart illustrating a procedure of the operation of the ultrasonic diagnosis apparatus according to the embodiment;

FIG. 4 is an explanatory view illustrating an example of a mark set in a three-dimensional image in the embodiment;

FIG. 5 is an explanatory view illustrating a concrete example of mark setting in the embodiment;

FIG. 6 is an explanatory view illustrating an operation example of rescanning in the embodiment;

FIG. 7 is an explanatory view explaining the rescanning operation in the embodiment together with a moving state of a probe;

FIG. 8 is an explanatory view illustrating another example of the rescanning operation; and

FIG. 9 is an explanatory view illustrating an example of the mark setting in a second embodiment.

### DETAILED DESCRIPTION

An ultrasonic diagnosis apparatus according to an embodiment includes: a transmission/reception section that transmits/receives an ultrasonic wave with respect to a subject through an ultrasonic probe; an image data generation section that processes a reception signal acquired by the transmission/reception section to generate two-dimensional ultrasonic images; an image display processing section that processes the two-dimensional ultrasonic images to generate a three-dimensional image; a display section that displays the image generated by the image display processing section; a mark setting section that sets a mark in a region of interest of the three-dimensional image; a storage section that stores mark information indicating a space region corresponding to the mark in the three-dimensional image; and a controller that controls to perform predetermined processing uses the mark information stored in the storage section, when the space region corresponding to the mark is scanned by the ultrasonic probe in rescanning operation for the subject.

Hereinafter, an ultrasonic diagnosis apparatus and a medical image processing apparatus according to embodiments will be described in detail with reference to the drawings. Throughout the accompanying drawings, the same reference numerals are used to designate the same components.

### First Embodiment

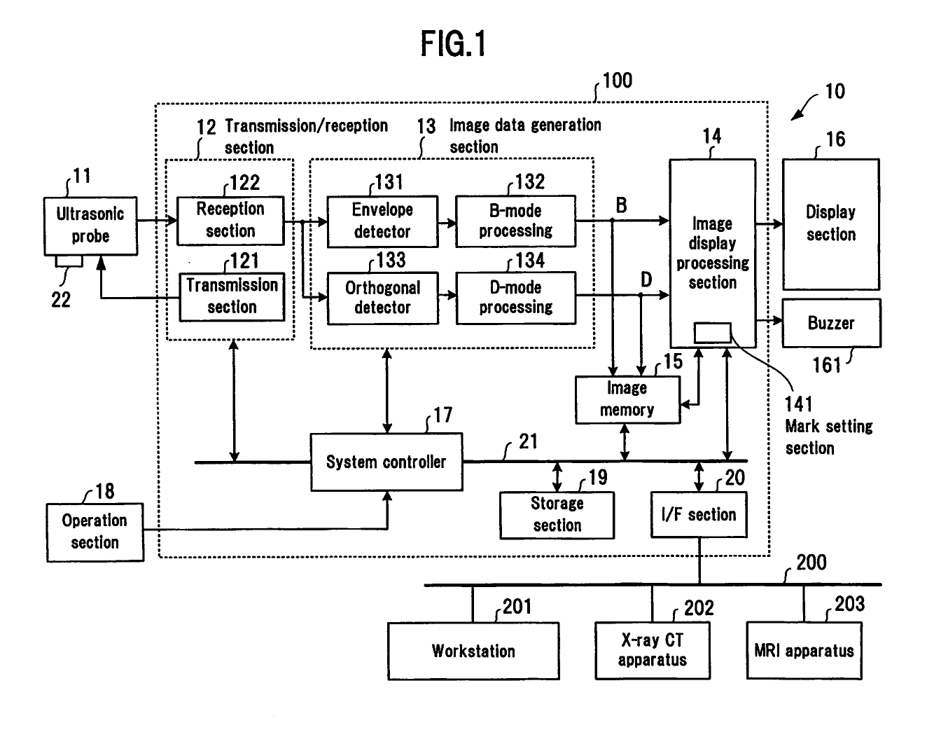

FIG. 1 is a block diagram illustrating a configuration of an ultrasonic diagnosis apparatus 10 as a medical image processing apparatus according to an embodiment. In FIG. 1, a main body 100 of the ultrasonic diagnosis apparatus 10 is connected with an ultrasonic probe 11 that transmits/receives an ultrasonic wave with respect to a subject (not illustrated). The main body 100 includes a transmission/reception section 12 that drives the ultrasonic probe 11 to perform ultrasonic scanning for the subject and an image data generation section 13 that processes a reception signal acquired by the transmission/reception section 12 to generate image data such as B-mode image data and Doppler image data.

The main body 100 includes an image display processing section 14 and an image memory 15. The image display processing section 14 is connected with a display section 16. The image display processing section 14 processes image data from the mage data generation section 13 to display in real time a two-dimensional ultrasonic image on the display section 16. Further, the image display processing section 14 generates a three-dimensional image from the two-dimensional image and display the generated three-dimensional image on the display section 16. The image memory 15 stores the image data generated by the image data generation section 13 and image data generated by the image display processing section 14.

The main body 100 further includes a system controller 17 that controls the entire apparatus. The system controller 17 is connected with an operation section 18 through which various command signals and the like are input. The main body 100 further includes a storage section 19 that stores mark information (to be described later) and an interface section (I/F section) 20 for connecting the main body 100 to a network 200. The I/F section 20 is connected, via the network 200, with a workstation (image processing section) 201 and a medical image diagnosis apparatus such as an X-ray CT apparatus 202 and an MRI apparatus 203. The system controller 17 and the above circuit sections are connected via a bus line 21.

The ultrasonic probe 11 transmits/receives an ultrasonic wave while bringing a leading end face thereof into contact with a body surface of the subject and has a plurality of piezoelectric vibrators arranged in one dimension. The piezoelectric vibrator is an electro-acoustic conversion element, which converts an ultrasonic driving signal into a transmitting ultrasonic wave at transmission and converts a receiving ultrasonic wave from the subject into an ultrasonic receiving signal at reception. The ultrasonic probe 11 is, e.g., an ultrasonic probe of a sector type, of a linear type, or of a convex type. The ultrasonic probe 11 is mounted with a sensor 22 that acquires position/angle information of the ultrasonic probe 11.

The transmission/reception section 12 includes a transmission section 121 that generates the ultrasonic driving signal and a reception section 122 that processes the ultrasonic receiving signal acquired from the ultrasonic probe 11. The transmission section 121 generates the ultrasonic driving signal and outputs it to the ultrasonic probe 11. The reception section 122 outputs the ultrasonic receiving signal acquired from the piezoelectric vibrators to the image data generation section 13. When an ultrasonic wave is transmitted from the ultrasonic probe 11 to the subject, the transmitted ultrasonic wave is sequentially reflected by a discontinuous surface of acoustic impedance of internal body tissue and is received by the plurality of piezoelectric vibrators as a reflected wave signal.

The ultrasonic probe 11 in the embodiment may be a one-dimensional ultrasonic probe in which a plurality of piezoelectric vibrators are arranged in one row so as to scan the subject two-dimensionally or in which the plurality of piezoelectric vibrators are mechanically swung. Alternatively, the ultrasonic probe 11 may be a two-dimensional ultrasonic probe in which a plurality of piezoelectric vibrators are two-dimensionally arranged in a matrix so as to scan the subject three-dimensionally.

The image data generation section 13 includes an envelope detector 131 and a B-mode processing section 132 that processes an output of the envelope detector 131. The image data generation section 13 further includes an orthogonal detector 133 and a Doppler mode (D-mode) processing section 134 that processes an output of the orthogonal detector 133.

The envelope detector 131 performs envelope detection for a reception signal from the reception section 122. The envelope detection signal is supplied to the B-mode processing section 132, and two-dimensional tomographic image data is acquired from the B-mode processing section 132 as a B-mode image. In the B-mode processing section 132, the signal that has been subjected to the envelop detection is logarithmically amplified, followed by digital conversion, to thereby acquire the B-mode image data.

The orthogonal detector 133 performs orthogonal phase detection for the reception signal supplied from the reception section 122 to extract a Doppler signal and supplies the extracted Doppler signal to the D-mode processing section 134. The D-mode processing section 134 detects a Doppler shift frequency of the signal from the transmission/reception section 12 and then converts the signal into a digital signal. After that, the D-mode processing section 134 extracts a blood flow or tissue and a contrast medium echo component based on Doppler effect, generates data (Doppler data) including mobile object information such as a mean speed, variance, power, and the like which are extracted at multiple points, and outputs the generated data to the image display processing section 14.

The image display processing section 14 generates a two-dimensional ultrasonic image for display using the B-mode image data, Doppler image data, and the like output from the image data generation section 13. Further, the image display processing section 14 generates a three-dimensional image from the two-dimensional ultrasonic image and displays the generated three-dimensional image on the display section 16. The image memory 15 stores the image data generated by the image display processing section 14. When review is made after inspection, the image data stored in the image memory 15 is read out and displayed on the display section 16. The image display processing section 14 includes a mark setting section 141.

The system controller 17 has a CPU, a RAM, a ROM, and the like and executes various processing while controlling the entire ultrasonic diagnosis apparatus 10. The operation section 18 is an interactive interface provided with an input device such as a keyboard, a track ball, or a mouse and a touch command screen. The operation section 18 performs input of patient information or various command signals, setting of ultrasonic wave transmission/reception conditions, setting of generation conditions of various image data, and the like.

The system controller 17 controls, based on, e.g., various setting requests input through the operation section 18 or various control programs and various setting information read from the ROM, the transmission/reception section 12, B-mode processing section 132, Doppler processing section 134, and image display processing section 14. Further, the system controller 17 performs control so as to display the ultrasonic image stored in the image memory 15 on the display section 16. In addition to the display section 16, a buzzer 161 may be provided. The system controller 17 performs control so as to notify the operator of various messages through the display section 16 or buzzer 161. The display section 16 may be controlled so as to display a scan direction of the ultrasonic probe 11. For example, a scan direction in the previous scanning may be displayed for guidance.

The I/F section 20 is an interface for exchanging various information between the network 200 and main body 100. The system controller 17 exchanges three-dimensional image data with another medical image diagnosis apparatus (X-ray CT apparatus 202, MRI apparatus 203, etc.) via the network 200 according to, e.g., DICOM (Digital Imaging and Communications in Medicine) protocol. The workstation 201, which constitutes an image processing section, acquires the three-dimensional image data (volume data) from the ultrasonic diagnosis apparatus 10 and processes the acquired volume data.

Further, the system controller 17 performs alignment between an arbitrary cross section of the three-dimensional image data generated by the X-ray CT apparatus 202, MRI apparatus 203, etc. and a cross section to be scanned by the ultrasonic probe 11 to thereby associate the three-dimensional image data with a three-dimensional space. As a result, when the subject is scanned by the ultrasonic probe 11, a CT image or an MRI image in which focus of disease has been detected is displayed as a reference image to thereby allow alignment between a cross section to be scanned and the reference image.

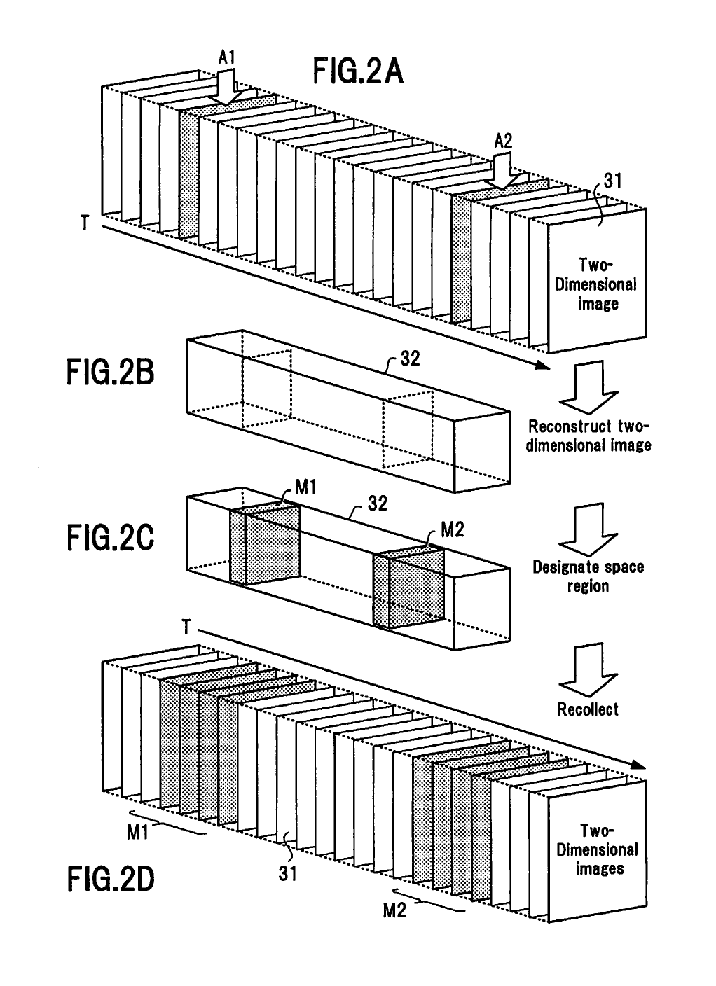

The following describes operation of the ultrasonic diagnosis apparatus according to the embodiment with reference to FIGS. 2A to 2D. FIGS. 2A to 2D are explanation views illustrating a basic operation of the embodiment. In the following description, the ultrasonic probe 11 is sometimes referred to merely as “probe 11”.

An operator (a doctor, an inspector, a surgeon, etc.) scans a subject while sweeping the probe 11 over the subject to thereby acquire a two-dimensional tomographic image.

FIG. 2A illustrates a set of two-dimensional tomographic images 31 acquired through scanning over a certain region. T denotes a time axis. Further, in FIG. 2A, when a region of interest (arrows A1 and A2, etc.) which is considered to be a diseased part (e.g., tumor site) exists, the operator preferably clicks a mouse of the operation section 18 to check the region of interest.

After completion of scanning over a certain region, the operator uses position information of the probe 11 acquired simultaneously with the scanning to construct a three-dimensional image 32 from continuous two-dimensional tomographic images 31 acquired by the sweeping of the ultrasonic probe 11.

FIG. 2B illustrates the three-dimensional image 32 constructed by stacking the continuous two-dimensional tomographic images 31.

Then, when the operator determines to perform rescanning for detailed check of the acquired three-dimensional image 32, he or she selects, from the acquired three-dimensional image 32, a position to be scanned in more detail, for example, a region of interest such as a tumor site and puts a mark on the selected position. The mark setting section 141 puts the mark on the region of interest such as the tumor site and sets a space region that surrounds the tumor site.

FIG. 2C illustrates marks M1 and M2 set in the three-dimensional image 32. The marks M1 and M2 are each set in a certain range including the previously checked position (A1 or A2). Portions with the marks M1 and M2 each correspond to a segment region that surrounds the tumor site that the operator has found. Information (position or size) of the space region of each of the marks M1 and M2 in the three-dimensional image that the operator has set is stored in the storage section 19 as mark information (segment information).

An arbitrary number of marks can be set. In FIG. 2C, two marks (M1 and M2) are set. The mark information may be associated with patient data and stored in the storage section 19.

Then, the operator rescans the subject. At this time, when an ultrasonic beam of the probe 11 enters the segment region denoted by the mark M1 or M2, the system controller 17 displays information on a screen of the display section 16 so as to make the operator understand that the ultrasonic beam has entered the segment region. This allows the operator to understand that the region denoted by the mark M1 or M2 is being scanned. For more detailed scanning, the operator may slow down a moving speed of the probe 11.

FIG. 2D illustrates a set of the two-dimensional tomographic images acquired by the rescanning. In FIG. 2D, portions corresponding to the space regions denoted by the marks M1 and M2 are illustrated in a different color.

After completion of the detailed scanning, a three-dimensional image is automatically constructed in the same manner as in the previous scanning. The operator confirms the constructed three-dimensional image. If the operator is not satisfied with the image, he or she performs the scanning once again to repeat the above procedure. When determining that satisfactory images corresponding respectively to the plurality of set segment regions have been acquired, the operator ends the scanning.

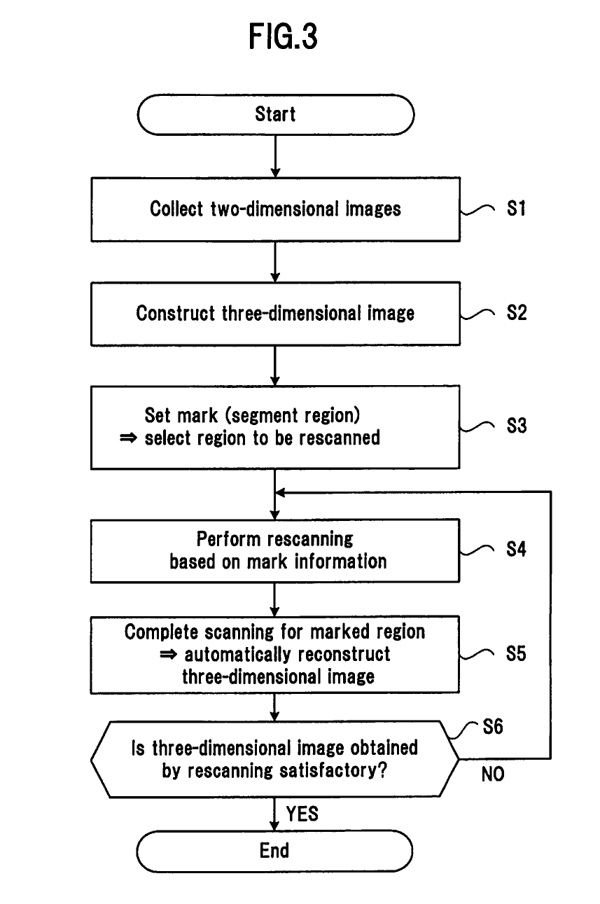

FIG. 3 is a flowchart illustrating a procedure of the above operation. In step S1 of FIG. 3, the subject is scanned with the probe 11 swept over the subject, to thereby acquire the two-dimensional tomographic images. In step S2, the three-dimensional image is constructed from the continuous two-dimensional tomographic images acquired by the sweeping.

In subsequent step S3, the mark is set in a position to be scanned in more detail so as to select the segment region to be rescanned in detail. In step S4, the rescanning is performed according to the mark information. In the rescanning, the marked region is scanned in more detail.

In step S5, after completion of the rescanning for the marked segment region, the three-dimensional image is automatically reconstructed. In step S6, the operator determines whether or not the three-dimensional image acquired by the rescanning is satisfactory. When it is determined that the acquired three-dimensional image is not satisfactory, the operation of step S4 is performed once again. Alternatively, according to need, the mark may be reset in step S3. When it is determined that satisfactory images of the plurality of selected segment regions have been acquired, the scanning is ended.

The operator can store the reconstructed three-dimensional image at an arbitrary timing. In a case where a plurality of scan data corresponding to the same segment exist as a result of a plurality of rescanning operations (more detailed scanning operations) performed in step S4, the operator can select the data to be stored from the plurality of data. Further, in a case where a plurality of segment regions selected in step S3 exist and where a plurality of data corresponding to each segment exist, the operator can select a plurality of data to be stored.

There may be a case where when a patient for whom the mark is set is subjected to rescanning at another inspection, an operator desires to acquire once again a three-dimensional image corresponding to the previous segment region. In this case, the operator can read out the mark information stored in the storage section 19 by means of switching operation and thus can use the two-dimensional images obtained by scanning for the space region corresponding to the mark information, to thereby construct the three-dimensional image.

Further, the mark can be set by means of the workstation 201. In this case, the two-dimensional images or three-dimensional images stored in the image memory 15 of the ultrasonic diagnosis apparatus 10 are loaded into the workstation 201 and processed therein so as to allow the workstation 201 to set the mark. The mark information set in the workstation 201 is stored in the storage section 19 of the ultrasonic diagnosis apparatus 10. When rescanning is performed, the mark information stored in the storage section 19 is used. That is, in this case, the workstation 201 constitutes the mark setting section.

Further, the position/angle sensor 22 is mounted to the probe 11, so that the operator can know scanning start position and scanning angle in the previous inspection. Therefore, by recording the position information of the probe in the image memory 15 together with the two-dimensional tomographic images and reading out the recorded information, the same region can be scanned in the subsequent scanning.

Further, when the operator finds a portion that he or she is concerned about after acquisition of the two-dimensional image or three-dimensional image as a result of the first inspection (scanning) by the ultrasonic diagnosis apparatus 10, rescanning may be performed immediately with a mark set to the concerned portion. In this case, the second scanning is executed for a segment region indicated by the set mark, that is, more detailed scanning is performed. The position information of the probe 11 in the first scanning can be recorded in the image memory 15 or the like. In this case, when the second scanning is performed, the same region can be scanned by reading out the stored probe information.

That is, by storing information indicating position, angle, depth, etc. of the probe 11 together with the mark information, an imaging setting and the like for rescanning can be set in the same manner as in the first scanning. In this case, when the probe approaches the set mark, a guide is displayed, and the three-dimensional images are collected while the segment region is scanned. Further, by setting an activation action for each segment, rescanning can be performed quickly.

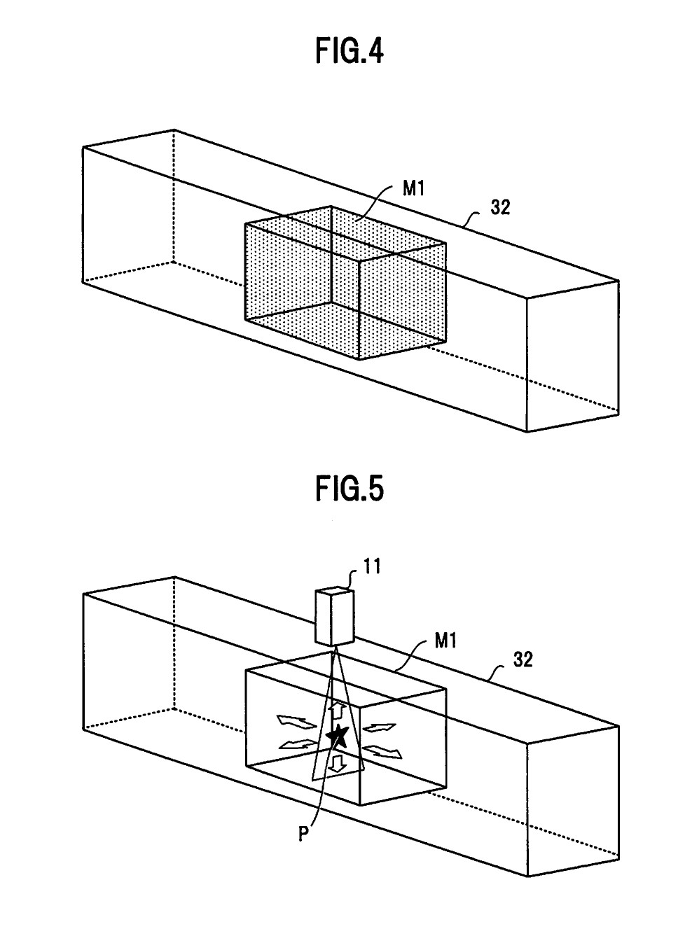

The following concretely describes the mark setting. A size and a position of the mark can be set by the operator operating the operation section 18. That is, as illustrated in FIG. 4, the operator designates, within a space of the collected three-dimensional image, a segment region to be recollected and sets a mark M1 to the designated segment region. For example, MPR (Multi Planar Reconstruction) is known as three-dimensional image processing, in which the mark is set in an MPR image that can be viewed in three axes.

Alternatively, by selecting a region of interest in the two-dimensional tomographic image with a pointer or the like, it is possible to automatically set the mark in a region of a previously set range. For example, assume that there exist a region (tumor site, etc.) to be checked in more detail in an image acquired by the first scanning, as indicated by A1 and A2 of FIG. 2A. In this case, the operator operates the operation section 18 to select a two-dimensional tomographic image (frame) in which the region of interest exists and designates a point of interest (P represented by a star mark), as illustrated in FIG. 5. Then, a space of a previously set certain range around the interest point P is automatically calculated, and the mark M1 having a prescribed size is generated.

Then, the mark information indicating the position and size of the mark M1 is stored in the storage section 19. At this time, the size of the mark M1 is determined according to, e.g., a program stored in the ROM included in the system controller 17. Further, the size of the mark may be previously set for each region to be inspected.

Thus, by setting the interest point P of the frame and mark M1 indicating the segment region in the three-dimensional image, it is possible to designate the region of interest. When there exist a plurality of regions of interest, the marks may be displayed so as to be identifiable from each other. For example, the mark M1 indicating the first segment is displayed in red, and mark M2 indicating the second segment is displayed in blue. Further, a position of the mark may be displayed on a body mark representing a whole body so as to make the operator easily understand where the mark exists within the whole body. Further, the mark position may be displayed with the body marks or characters made different for each region of interest.

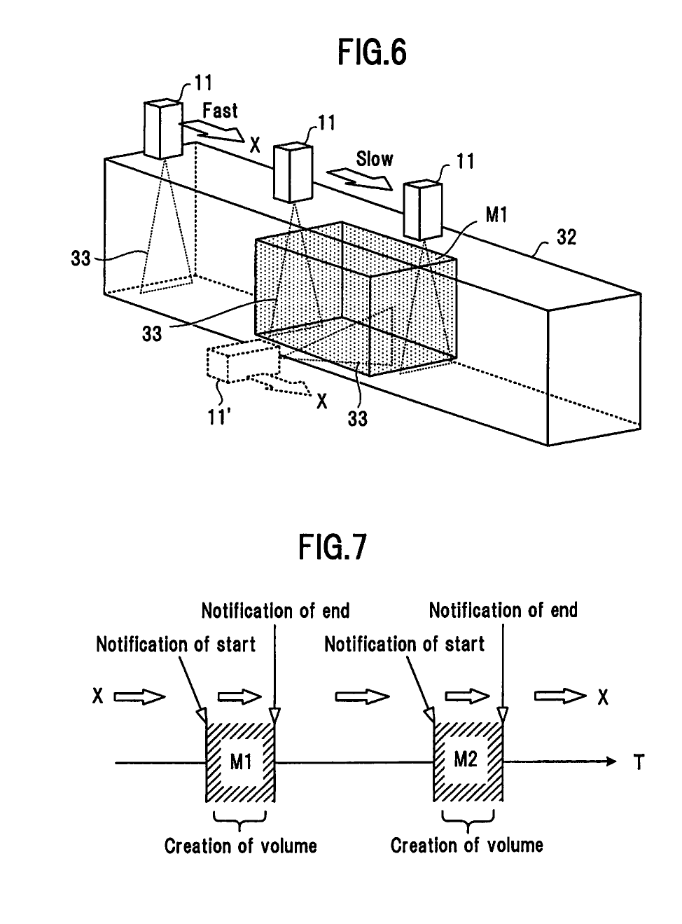

FIGS. 6 and 7 are explanation views illustrating an example of rescanning operation performed for the marked segment region. FIG. 6 is a view illustrating rescanning operation performed for the segment region corresponding to the mark M1 set in FIG. 5. FIG. 7 is a view illustrating rescanning operation performed for the segment regions corresponding respectively to the marks M1 and M2 with the probe 11 being moved in an X-arrow direction.

In the rescanning operation in FIGS. 6 and 7, the same region of the same subject as that in the previous scanning is scanned based on the position information of the probe 11 obtained in the previous scanning. Further, a scan direction in the previous scanning can be used as a guide for rescanning if it is displayed. When the probe 11 is swept over the subject, and an ultrasonic beam 33 of the probe 11 enters a position corresponding to the mark M1, the system controller 17 performs control such that predetermined processing is executed using the mark information stored in the storage section 19. The predetermined processing includes a message notification, reconstruction of the three-dimensional image, and the like.

For example, when the ultrasonic beam 33 of the probe 11 enters the position corresponding to the mark M1, the system controller 17 makes a notification indicating start of the scanning for the region of interest through a message saying “enter region of interest” displayed on the display section 16 or through a sound such as the buzzer 161.

When the probe 11 enters the segment region indicated by the mark M1, the probe 11 is decelerated for detailed scanning so that high-resolution image can be obtained. When the probe 11 goes out of the region indicated by the mark M1, a message saying, e.g., “outside region of interest” indicating end of the scanning for the region of interest is displayed, followed by transition to normal scanning. Further, as denoted by a broken line (probe 11′) in FIG. 6, the scan direction may be changed. In this case, as in the above example, the probe 11 is swept in the X-arrow direction and, when the ultrasonic beam 33 enters the region corresponding to the mark M1 and when it goes out thereof, the message is displayed for the operator.

Further, while the probe 11 enters the segment region indicated by the mark M1, a message indicating that the probe 11 is within the region of the mark M1 may be displayed.

When the mark M2 is set in addition to the mark M1, the same operation as that for the region of the mark M1 is performed. That is, as illustrated in FIG. 7, detailed scanning is performed for the segment indicated by the mark M2, followed by the notification. Note that it is possible to perform the scanning for the region of the mark M1 (M2) in an opposite direction to the X-arrow direction in FIG. 7. Also in this case, the message is displayed for the operator when the ultrasonic beam 33 enters the region corresponding to the mark M2 (M1) and when it goes out thereof.

Further, the system controller 17 performs reconstruction of the three-dimensional image as the above-mentioned predetermined processing. That is, while the operator performs detailed scanning for the regions of the marks M1 and M2, the system controller 17 performs, in real time, reconstruction (creation of volume data) of the three-dimensional image from the collected two-dimensional tomographic images and displays a state of the reconstruction on a screen of the display section 16. This makes the operator easily understand the size of the space region he or she scanned.

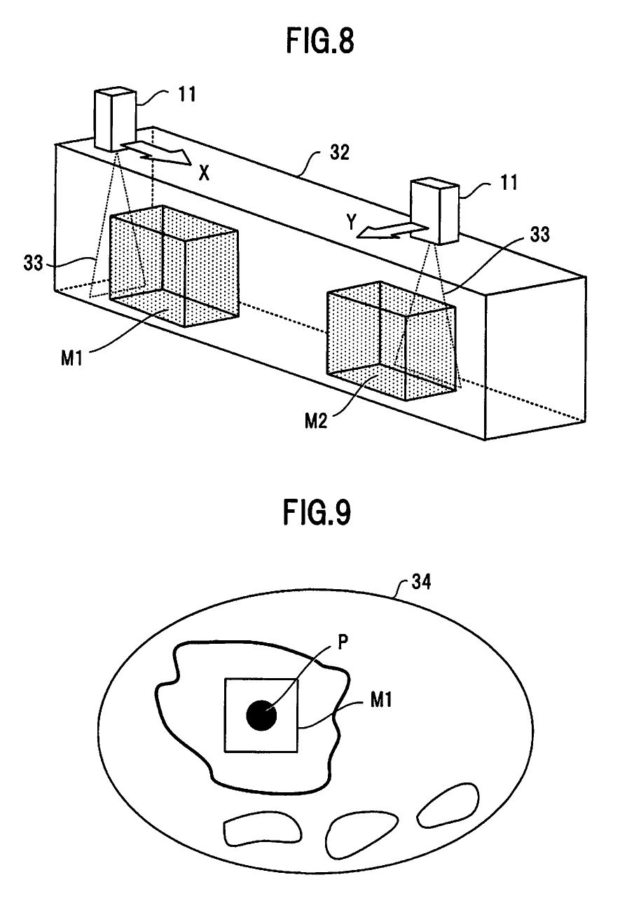

Further, as illustrated in FIG. 8, the probe 11 can be swept not only in the X-arrow direction, but also in, e.g., a Y-arrow direction which is perpendicular to the X-arrow direction. In FIG. 8, the segment region corresponding to the mark M1 is rescanned in the X-arrow direction, and segment region corresponding to the mark M2 is rescanned in the Y-arrow direction.

The message is displayed when the ultrasonic beam 33 of the probe 11 enters the region of the mark M1 in the X-arrow direction and when it goes out thereof in the X-arrow direction; similarly, the message is displayed when the ultrasonic beam 33 of the probe 11 enters the region of the mark M2 in the Y-arrow direction and when it goes out thereof in the Y-arrow direction. That is, the notification to the operator is made for each of the marks M1 and M2.

There may be a case where it is not necessary to perform the reconstruction of the three-dimensional image from the two-dimensional tomographic images when the rescanning is performed based on the mark information stored in the storage section 19. To cope with such a case, the mark can be set “enable” or “disable” by operator's operation. When the mark is set “disable”, reconstruction of the three-dimensional image is automatically stopped after the probe 11 completes the scanning for the marked region.

Further, the operator can perform operation of editing, deleting, etc., the mark information stored in the storage section 19. For example, the operator can delete unnecessary mark information or change the size or position of the mark.

### Second Embodiment

The mark can be set not only using the ultrasonic diagnosis apparatus 10, but also using an arbitrary three-dimensional image in another medical image diagnosis apparatus such as the X-ray CT apparatus 202 or MRI apparatus 203. In the second embodiment, the point P of interest is designated by another medical image diagnosis apparatus, then a space region of a previously set certain range is automatically calculated with the designated point P as a center, and the mark M1 having a prescribed size is generated.

That is, the system controller 17 aligns an arbitrary cross section of the three-dimensional image data generated by the X-ray CT apparatus 202 or MRI apparatus 203 and a cross section to be scanned by the ultrasonic probe 11 to thereby associate the three-dimensional image data with three-dimensional space. In a case of using a CT image in the alignment, by making positions (more than four place) of xiphoid process, rib, base of umbilicus, kidney coincide with each other, it is possible to make the positions of the CT image and probe 11 coincide with each other unless a body is moved.

FIG. 9 is an explanatory view illustrating an example of the mark setting in the second embodiment. For example, as illustrated in FIG. 9, simply by designating a point P of interest in a CT image 34 in which focus of disease has been detected, a mark M1 can be set. When scanning the subject with the probe 11, the ultrasonic diagnosis apparatus 10 applies the mark M1 set by the X-ray CT apparatus 202 and scans the same region of the subject as that photographed by the X-ray CT apparatus while sweeping the probe 11 over the subject.

Then, when the probe 11 enters the segment region indicated by the mark M1 set using the CT image 34, a notification that scanning for the region of interest is made, making it possible to prompt the operator to perform detailed scanning. The subsequent steps are the same as steps S4 to S6 in FIG. 3.

According to at least one of the above-described embodiment, the mark set in the three-dimensional image can be used as an index for moving the probe to the region of interest in the subsequent rescanning. Further, when the three-dimensional image data corresponding to the region of interest needs to be acquired once again, start/stop positions can be notified automatically for each region of interest, so that it is possible to ensure reproducibility of image collection start/end positions.

While certain embodiments have been described, these embodiments have been presented by way of example only, and are not intended to limit the scope of the invention. Indeed, the novel apparatus and methods described herein may be embodied in a variety of other forms; furthermore, various omissions, substitutions and changes in the form of the apparatus described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus, comprising:
a transmission/reception section that transmits/receives an ultrasonic wave with respect to a subject through an ultrasonic probe;
an image data generation section that processes a reception signal acquired by the transmission/reception section to generate two-dimensional ultrasonic images;
an image display processing section that processes the two-dimensional ultrasonic images to generate a three-dimensional image;
a display section that displays the image generated by the image display processing section;
a mark setting section that sets a mark in a region of interest of the three-dimensional image;
a storage section that stores mark information indicating a space region corresponding to the mark in the three-dimensional image; and
a controller that controls to perform predetermined processing uses the mark information stored in the storage section, when the space region corresponding to the mark is scanned by the ultrasonic probe in rescanning operation for the subject.

2. The apparatus of claim 1, wherein
the controller makes a notification indicating that a rescanning region of the ultrasonic probe enters the space region corresponding to the mark, as the predetermined processing.

3. The apparatus of claim 1, wherein
the controller controls the image display processing section to reconstruct the three-dimensional image from continuous two-dimensional images included in the space region corresponding to the mark, as the predetermined processing.

4. The apparatus of claim 1, wherein
when a point of interest is designated in the three-dimensional image displayed on the display section, the mark setting section automatically sets a region of a predetermined range from a point of interest as the space region corresponding to the mark.

5. The apparatus of claim 1, further comprising:
a notification section a notification indicating that an ultrasonic beam of the ultrasonic probe enters the space region corresponding to the mark and that the ultrasonic beam goes out of the space region corresponding to the mark, when the rescanning an inspection region of the subject including the mark by the ultrasonic probe.

6. The apparatus of claim 1, wherein
the mark setting section can edit the mark setting.

7. The apparatus of claim 1, wherein
the ultrasonic probe includes a sensor that acquires position information, and
the image display processing section aligns an arbitrary cross section of the three-dimensional image and a cross section to be scanned by the ultrasonic probe based on the position information of the ultrasonic probe when the rescanning, and to reconstruct a three-dimensional image based on the rescanning.

8. A medical image processing apparatus comprising:
an image display processing section that processes two-dimensional ultrasonic images of a subject to generate a three-dimensional image;
a display section that displays the image generated by the image display processing section;
a mark setting section that sets a mark in a region of interest of the three-dimensional image;
a storage section that stores mark information indicating a space region corresponding to the mark in the three-dimensional image; and
a controller that controls to perform predetermined processing uses the mark information stored in the storage section, when the space region corresponding to the mark is scanned by an ultrasonic probe in rescanning operation for the subject.

9. The apparatus of claim 8, wherein
the controller makes a notification indicating that a rescanning region of the ultrasonic probe enters the space region corresponding to the mark, as the predetermined processing.

10. The apparatus of claim 8, wherein
the controller controls the image display processing section to reconstruct the three-dimensional image from continuous two-dimensional images included in the space region corresponding to the mark, as the predetermined processing.

11. The apparatus of claim 8, wherein
when a point of interest is designated in the three-dimensional image displayed on the display section, the mark setting section automatically sets a region of a predetermined range from a point of interest as the space region corresponding to the mark.

12. The apparatus of claim 8, wherein
the mark setting section can edit the mark setting.

13. The apparatus of claim 8, wherein
the image display processing section is provided in an ultrasonic diagnosis apparatus, and
the mark setting is performed by an image processing section connected to the ultrasonic diagnosis apparatus.

14. The apparatus of claim 8, wherein
the image display processing section is provided in an ultrasonic diagnosis apparatus,
the mark is set in an arbitrary cross section of the three-dimensional image generated by the medical image diagnosis apparatus connected to the ultrasonic diagnosis apparatus, and
the controller aligns the arbitrary cross section of the three-dimensional image generated by the medical image diagnosis apparatus and a cross section to be scanned by the ultrasonic probe, and controls the image display processing section to reconstruct a three-dimensional image based on the rescanning.
